(19) United States
(12) Patent Application Publication
(10) Pub. No.: US 2024/0070045 A1
(43) Pub. Date: Feb. 29, 2024

(54) CORRECTING APPLICATION BEHAVIOR USING USER SIGNALS PROVIDING BIOLOGICAL FEEDBACK

(71) Applicant: Microsoft Technology Licensing, LLC, Redmond, WA (US)

(72) Inventors: Kuan-Jung CHIANG, San Diego, CA (US); Weiwei YANG, Seattle, WA (US); Hayden Sullivan HELM, San Francisco, CA (US); Amber Dawn HOAK, Silverdale, WA (US)

(21) Appl. No.: 17/897,841

(22) Filed: Aug. 29, 2022

(51) Int. Cl. G06F 11/34; G06N 20/00

(52) U.S. Cl. CPC G06F 11/3438; G06N 20/00

(57) ABSTRACT

The present disclosure relates to methods and systems for improving user experiences of an application. The methods and systems receive biological feedback of the user as the user interacts with an application and adapts the application's behavior in response to detecting a user's instantaneous reaction in response to interactions with the application. The methods and systems use a machine learning model to compare the user's current state based on the biological feedback received to known states to determine the user's instantaneous reaction to interactions with the application. The methods and systems provide feedback with a user experience classification of the user's interactions with the application.

### BACKGROUND

When users interact with applications on a device, it is possible that the application does not behave as expected. These experiences can make the user frustrated and, in some cases, quit using the application entirely.

### BRIEF SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Some implementations relate to a method. The method includes receiving a plurality of processed signals providing biological feedback of a user interacting with an application. The method includes determining to take an action in the application in response to analyzing the plurality of processed signals. The method includes providing feedback in response to taking the action in the application.

Some implementations relate to a system. The system includes a processor; memory in electronic communication with the processor; and instructions stored in the memory, the instructions being executable by the processor to: receive a plurality of processed signals providing biological feedback of a user interacting with an application; determine to take an action in the application in response to analyzing the plurality of processed signals; and provide feedback in response to taking the action in the application.

Some implementations relate to a method. The method includes receiving feedback indicating that an undesirable user experience occurred in an application, wherein the feedback is based on an analysis of biological feedback of a user interacting with the application. The method includes correcting the user experience of the application in response to receiving the feedback.

Some implementations relate to a system. The system includes a processor; memory in electronic communication with the processor; and instructions stored in the memory, the instructions being executable by the processor to: receive feedback indicating that an undesirable user experience occurred in an application, wherein the feedback is based on an analysis of biological feedback of a user interacting with the application; and correct the user experience of the application in response to receiving the feedback.

Additional features and advantages will be set forth in the description which follows, and in part will be obvious from the description, or may be learned by the practice of the teachings herein. Features and advantages of the disclosure may be realized and obtained by means of the instruments and combinations particularly pointed out in the appended claims. Features of the present disclosure will become more fully apparent from the following description and appended claims or may be learned by the practice of the disclosure as set forth hereinafter.

### BRIEF DESCRIPTION OF THE DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 illustrates an example of a user and the different signals obtained from the user in accordance with implementations of the present disclosure.

FIG. 2 illustrates an example environment for detecting user experiences of an application using user signals and correcting undesirable user experiences of an application in accordance with implementations of the present disclosure.

FIGS. 3A-3C illustrate an example user interface of an undesirable user experience that occurred in an application in accordance with implementations of the present disclosure.

FIG. 4 illustrates an example method for determining that an undesirable user experience occurred in an application in accordance with implementations of the present disclosure.

FIG. 5 illustrates an example method for correcting a user experience in an application in accordance with implementations of the present disclosure.

FIG. 6 illustrates components that may be included within a computer system.

### DETAILED DESCRIPTION

This disclosure generally relates to interactions with applications by users. When users interact with applications on a device, it is possible that the application does not behave as expected. A UI error may occur as the user is interacting with the application. For example, a UI error includes a scroll bar not scrolling. Another example of a UI error includes a button not clicking. Another example of a UI error includes highlighting an incorrect block of text. As such, a UI error occurs when the application makes a functional error.

Errors may also occur with the user experience (UX) of applications anticipating what the user is doing. The user experience errors include unexpected or bad interactions with the application. The user experience errors may occur even though the UI is functional and performing correctly. For example, a user experience error occurs if the user asked a digital assistance to call “Greg,” and the digital assistance calls “Craig.” For example, a user experience provides type suggestions or auto complete suggestions for use with the applications. However, the auto complete suggestions do not always provide the best suggestion for the context. The auto complete suggestions are typically based on natural language processing and historical use of the users. As such, the natural language processing sometimes makes mistakes or is not correct in the current context of use. These experiences can make the user frustrated and, in some cases, quit using the application entirely.

The present disclosure relates to systems and methods for detecting the user's subtle reaction to unexpected behaviors of the application from biological feedback of the user interacting with an application and correcting the application's behavior in response to detecting the user's reaction. The systems and methods combine at least two streams of information (or data) to assess the user's reaction to the application's behavior. The first stream of information is based on the user's brain activities. In some implementations, a brain-computer Interface (BCI) obtains the user's brain activities. In particular, the systems and methods monitor the user's brain waves (e.g., via electroencephalogram (EEG) or a functional near-infrared spectroscopy (fNIRS)). The second stream of information is based on information that can be derived from digital sensing inputs, such as web cameras, cameras on devices, conductance, and/or breaking monitors. This information may be facial expressions or other biological-physical signals. Examples of the biological-physical signals include heart rate, heart rate variability, skin temperature, breathing rate, blood oxygen level, pupil dilation, and/or gaze tracking. As such, the streams of data provide biological feedback of the user as the user interacts with the application.

In some implementations, each of the streams of data (e.g., the user's brain activities and the biological-physical signals) may be processed independently and used as input to individual machine learning (ML) models. In some implementations, the streams of data (e.g., the user's brain activities and the biological-physical signals) may be processed jointly and used as input to a multi-modal ML model. In either case, the ML model is used to compare the user's current state to known states of surprise, agitation, frustration, pleased, content, happy, or indifferent to determine a user's instantaneous reaction after encountering an UI interaction with the application. The systems and methods use the user's instantaneous reaction to determine that an undesirable user experience occurred in the UI interaction with the application and provide feedback indicating that the undesirable user experience occurred in the application based on the user's instantaneous reaction.

One example use case of the systems and methods of the present disclosure is to detect whether a text completion or autocorrection in an e-mail application is correct or matches a user's expectation based on the analysis of the streams of information (e.g., the biological feedback) obtained from the user. Another example use case of the systems and methods of the present disclosure is to revert unwanted changes or deletion of text in a document application in response to a user mistakenly highlighting the wrong section of a piece of text or inserting content in an unexpected location based on the analysis of the streams of information (e.g., the biological feedback) obtained from the user.

Another example use case of the systems and methods of the present disclosure is to automatically recall sent messages in an e-mail application or any other messaging application in response to a user accidentally pushing the send button based on the analysis of the streams of information (e.g., the biological feedback) obtained of the user.

Another example use case of the systems and methods of the present disclosure is to automatically reject programming keywords, functions or variables as the user is using integrated coding environments, improving coding productivity based on analysis of the streams of information (e.g., the biological feedback) of the user. Another example use case of the systems and method of the present disclosure is rejecting erroneous auto fills in data entry programs based on analysis of the streams of information (e.g., the biological feedback) obtained from the user.

One technical advantage of the systems and methods of the present disclosure is the improvement of the effectiveness of the error-correcting systems of the applications by incorporating user-specific biological signals (e.g., bodily signals and/or physiological signals) to personalize the error-correcting systems of the applications. Another technical advantage of the systems and methods of the present disclosure is reducing machine interjected errors in programming. Another technical advantage of the systems and methods of the present disclosure is reducing miscommunications in written documents.

Another technical advantage of the systems and methods of the present disclosure is improvement of the accessibility of the applications by making corrections automatically in the application without the user having to make the correction. As such, if the user has limited mobility or an accessibility issue, the systems and methods of the present disclosure improve the accessibility of the applications to the users by automatically making corrections in the application based on the biological feedback of the users. Another technical advantage of the systems and methods of the present disclosure is information collection of how the users interact with the applications to improve the applications.

As such, the systems and methods of the present disclosure improve the user experience with applications, improve digital ergonomics, and allow more personalized interactions with applications by the user. The systems and methods take the user's physiological and cognitive signals in an integrated way and use the physiological and cognitive sensing of the user to provide personalized interactions with applications by the user.

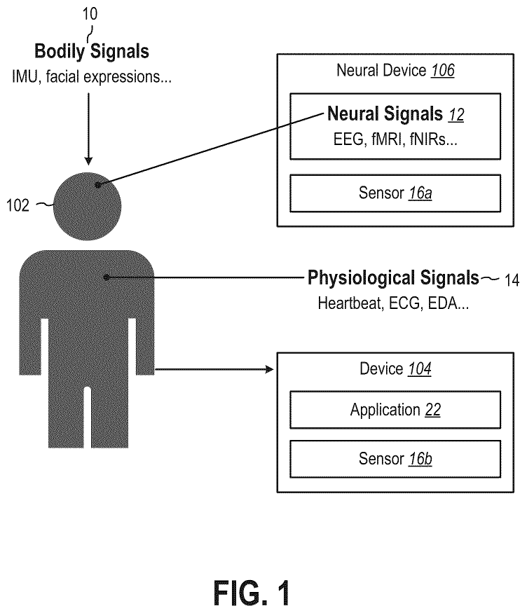

Referring now to FIG. 1, illustrated is an example user 102 interacting with one or more applications 22 using a device 104. The application 22 may be any kind of software that is designed to perform a group of coordinated functions, tasks, or activities for the benefit of the user.

Different signals are obtained from the user 102 interacting with the application 22 using the device 104. One type of signal obtained from the user 102 is bodily signals 10. The bodily signals 10 include signals relating to the user's physiological information. An example bodily signal 10 includes facial expressions. Another example bodily signal 10 includes pupil dilation. Another example bodily signal 10 includes gaze tracking. For example, a camera with one or more sensors (e.g., sensors 16a, 16b, up to 16n) on the device 104 captures the bodily signals 10 of the user 102. Another example includes an inertial measurement unit (IMU) to obtain the bodily signals 10 of the user 102.

Another type of signal obtained from the user 102 is neural signals 12. A neural device 106 is attached to the user 102. In some implementations, the neural device 106 picks up small variations in flux in the electro field as the neurons fire in the user's brain as the neural signals 12. In some implementations, the neural device 106 derives the neural signals from the magnetic or bloody flow in the user's brain. In some implementations, a Brain-computer Interface (BCI) is used to obtain the neural signals 12 of the user from the neural device 106. One example neural device 106 is an electroencephalogram (EEG) device where sensors on the EEG device obtains the neural signals 12 of the user 102 (e.g., EEG neural signals 12). Another example neural device 106 is a Magnetoencephalography (MEG) device where sensors on the MEG device obtain the neural signals 12 of the user 102 (e.g., MEG neural signals). Another example neural device 106 is an electromyography (EMG) device where sensors on the EMG device obtain the neural signals 12 of the user 102 (e.g., EMG neural signals). Another example neural device 106 is a functional magnetic resonance imaging (fMRI) device where sensors on the fMRI device obtain the neural signals 12 of the user 102 (e.g., fMRI neural signals). Another example neural device 106 is a functional near-infrared spectroscopy (fNIRS) device where sensors on the fNIRS device obtain the neural signals 12 of the user 102 (e.g., fNIRS neural signals).

Another type of signal obtained from the user 102 is physiological signals 14. One example physiological signal 14 includes a heart rate of the user 102. Another example physiological signal 14 includes skin temperature of the user 102. Another example physiological signal 14 includes core temperature of the user 102. Another example physiological signal 14 includes breathing rate of the user 102. Another example physiological signal 14 includes blood oxygen level of the user 102. Another example physiological signal 14 includes electrodermal activity (EDA) of the user's 102 skin. Different devices or sensors (e.g., sensors 16a, 16b, up to 16n) may be used to obtain the physiological signals 14 of the user 102. For example, an electrocardiogram (ECG or EKG) device is used to measure the heart rate of the user 102.

A plurality of signals (e.g., the bodily signals 10, the neural signals 12, and/or physiological signals 14), or any subset of the signals, are obtained from the user 102 during the interactions with the application 22 using the device 104. As such, the plurality of signals (e.g., the bodily signals 10, the neural signals 12, and/or physiological signals 14) provide biological feedback of the user 102 during the interactions with the application 22.

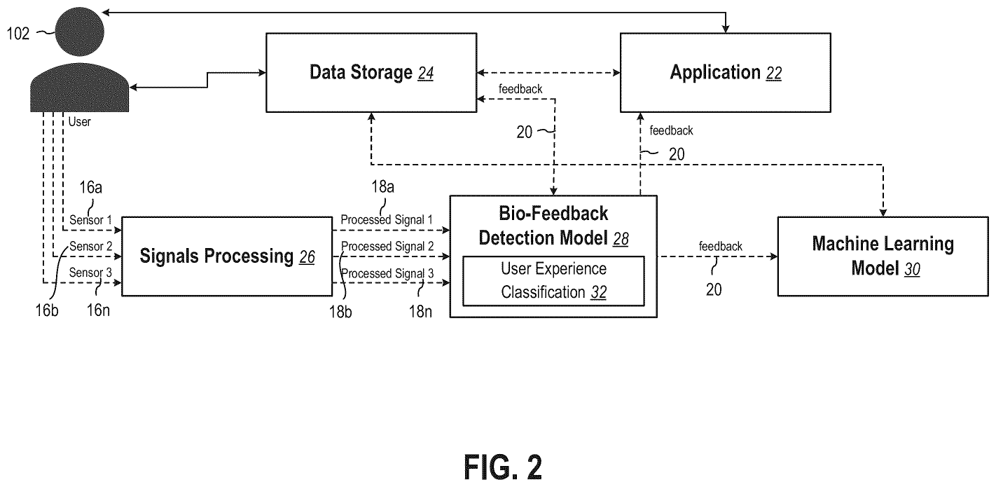

Referring now to FIG. 2, illustrated is an example environment 200 for detecting user experiences of an application 22 using user signals and correcting undesirable user experiences of the application 22. An undesirable user experience of an application includes UI errors of the application 22 and/or sub-optimal user experience with the application 22 (e.g., an unexpected or bad interaction with the application 22). The environment 200 includes one or more users 102 interacting with one or more applications 22 on a device 104 (FIG. 1). Interacting with the application 22 includes digital interactions with the application 22, audio (voice) interactions with the application 22, olfactory interactions with the application 22, or tactile interactions with the application 22. For example, the user 102 is accessing a messaging application. The application 22 provides a user interface or user experience for using the application 22. The user interface is how the user 102 interacts with the application 22. The user interface allows the user 102 to operate the application 22 to produce a desired result. The user interface makes it easy, efficient, and enjoyable (user-friendly) to operate the application 22 to produce a desired result. The user experience is how the user 102 interacts with the application 22 and experiences the application 22. The user experience includes the user's 102 perceptions of utility of the application 22, ease of use of the application 22, and efficiency of the application 22. The user experience is an overall experience of the user 102 interacting with the application 22. The application 22 includes different features that enhance the user experience. One example of the features that enhance the user experience of the application 22 is providing auto complete suggestions for use with the application 22. Another example of the features that enhance the user experience of the application 22 is providing auto fill suggestions for use with the application 22.

As the user 102 is interacting with the application 22, a plurality of sensors 16a, 16b up to 16n (where n is a positive integer) obtain signals from the user 102. The signals are the bodily signals 10 (FIG. 1), the neural signals 12 (FIG. 1), and/or the physiological signals 14 (FIG. 1) of the user 102. The signals may be any subset of the bodily signals 10, the neural signals 12, and/or the physiological signals 14. Each of the sensors 16a, 16b creates a data stream with the signals obtained from the users (e.g., the bodily signals 10, the neural signals 12, and/or the physiological signals 14).

In some implementations, the sensors 16a, 16b are in contact with the user 102 (e.g., EEG sensors, perspiration sensors, breathing rate sensors). In some implementations, the sensors 16a, 16b are non-contact sensors with the user 102 (e.g., cameras, eye tracking sensors, pupil dilation sensors). In some implementations, the sensors 16a, 16b are a combination of non-contact sensors and contact sensors with the user 102.

In some implementations, different devices or components include the sensors 16a, 16b. For example, a camera on the device 104 includes the sensor 16a and a wearable EEG device includes the sensor 16b. Another example includes a fNIR device includes the sensor 16a, an ECG device includes the sensor 16b, and a camera includes two sensors. As such, a plurality of sensors 16a, 16b up to 16n obtain a plurality of signals (e.g., the bodily signals 10, the neural signals 12, and/or the physiological signals 14) of the user 102 as the user 102 interacts with the application 22.

One or more signal processing components 26 process the obtained signals (e.g., the bodily signals 10, the neural signals 12, and/or the physiological signals 14) and outputs a plurality of processed signals 18a, 18b up to 18n (where n is a positive integer). In some implementations the signal processing component 26 is local to the device 104 of the user 102. In some implementations, the signal processing component 26 is remote from the device 104 on a remote server (e.g., a cloud server device). The signal processing component 26 processes the signals (e.g., the bodily signals 10, the neural signals 12, and/or the physiological signals 14) of the user 102 and outputs the processed signals 18a, 18b to use for downstream tasks.

In some implementations, the signal processing component 26 extracts features from the signals that are useful for a downstream tasks. An example of features for facial expressions include anchor points and a distance between anchor points in the user's 102 face. An example of features for brain waves includes distributions of the power band of alpha or gamma regions Another example of features for brain waves includes frontal alpha asymmetry. Another example of features for brain waves include channel correlation graphs. An example of features for heart rate is variable heart rate. As such, different information of the obtained signals is extracted and provided in the processed signals 18a, 18b. The processed signals 18a, 18b are provided to the biological feedback detection model 28 as input.

A biological feedback detection model 28 analyzes the plurality of processed signals 18a, 18b and determines user experience classification 32 of the application 22 in response to analyzing the processed signals 18a, 18b. In some implementations, the user experience classification 32 includes a positive user experience of the application 22. In some implementations, the user experience classification 32 includes a normal user experience of the application 22 (e.g., the user interface of the application 22 functioned properly or the user experience provided an expected result). In some implementation, the user experience classification 32 includes an undesirable user experience. An undesirable user experience includes a UI error or a sub-optimal user experience of the application 22. A UI error occurs when the application 22 makes a functional error in the user interface of the application 22. A sub-optimal user experience of the application 22 includes an unexpected or bad interaction with the application 22. A sub-optimal user experience of the application 22 includes any unexpected behaviors of the application 22. The biological feedback detection model 28 outputs feedback 20 in response to determining the user experience classification 32 of the application 22. The biological feedback detection model 28 may also provide the feedback 20 to the application 22 indicating the user experience classification 32 (e.g., a positive user experience, an undesirable user experience, or a normal user experience) of the user's 102 interactions with the application 22 based on analyzing the processed signals 18a, 18b.

The biological feedback detection model 28 is a machine learning model that receives the processed signals 18a, 18b as input and outputs the feedback 20. In some implementations, the biological feedback detection model 28 operates on the device 104 of the user 102 (e.g., the device the user 102 is using to access the application 22). In some implementations, the biological feedback detection model 28 is in a cloud server device remote from the device 104 of the user 102. In some implementations, the biological feedback detection model 28 is local to the device 104 of the user 102. In some implementations, the biological feedback detection model 28 has components that are both local to the device 104 of the user 102 and remote from the device 104 of the user 102 (e.g., in a cloud server device).

In some implementations, the biological feedback detection model 28 is trained offline with a large set of data across multiple users prior to being deployed to the device 104 or the remote server. In some implementations, the biological feedback detection model 28 is trained online with the feedback 20. In some implementations, the biological feedback detection model 28 is trained offline and online. The biological feedback detection model 28 is trained to identify the information in the plurality of processed signals 18a, 18b that indicates a current state of the user 102. In some implementations, the information is features in the plurality of processed signals 18a, 18b. The biological feedback detection model 28 compares the current state of the user 102 to a known state to determine an instantaneous reaction of the user 102 as the user 102 interacts with the application 22. Examples of a known state include frustration, surprise, agitation, anger, pleased, content, happy, or indifferent. The biological feedback detection model 28 uses the features in the processed signals 18a, 18b to determine a current state of the user and determine whether the instantaneous reaction of the user 102 is frustrated, surprised, agitated, angry, pleased, content, happy, and/or indifferent.

One example includes the biological feedback detection model 28 analyzing the anchor points in the facial expression of the user 102 to determine that the user 102 is frustrated. Another example includes the biological feedback detection model 28 analyzing the distributions of the power band of the processed neurological signal to determine that the user 102 is surprised. Another example includes the biological feedback detection model 28 analyzing the change in heart rate (heart rate variability (HRV)) of the processed physiological signals to determine that the user 102 is agitated. Another example includes the biological feedback detection model 28 analyzing the anchor points in the facial expression of the user 102 and the change in heart rate (HRV) to determine that the user 102 is happy. Another example includes the biological feedback detection model 28 analyzing the distribution of the power band of the processed neurological signals and the anchor points in the facial expression of the user 102 to determine that the user 102 is content. Another example includes the biological feedback detection model 28 analyzing the distribution of the power band of the processed neurological signals and the anchor points in the facial expression of the user 102 to determine that the user 102 is indifferent.

The biological feedback detection model 28 uses the instantaneous reaction of the user 102 to determine a user experience classification 32 of the user's 102 interactions with the application 22 and provide feedback 20 with the user experience classification 32. In some implementations, the user experience classification 32 indicates that an undesirable user experience occurred in the application 22. One example includes if the instantaneous reaction of the user 102 is frustrated, the biological feedback detection model 28 determines that an undesirable user experience occurred in the application 22 based on determining that the user is frustrated. Another example includes if the instantaneous reaction of the user 102 is surprised, the biological feedback detection model 28 determines that an undesirable user experience occurred in the application 22 based on determining that the user 102 is surprised. Another example includes if the instantaneous reaction of the user 102 is agitated, the biological feedback detection model 28 determines that an undesirable user experience occurred in the application based on determining that the user 102 is agitated. The biological feedback detection model 28 provides feedback 20 in response to determining that an undesirable user experience occurred in the application based on the analysis of the instantaneous reaction of the user 102. One example includes if the instantaneous reaction of the user 102 is happy, the biological feedback detection model 28 determines that a positive user experience occurred in the application 22 based on determining that the user 102 is happy. The biological feedback detection model 28 provides feedback 20 in response to determining that a positive user experience occurred in the application based on the analysis of the instantaneous reaction of the user 102. One example includes if the instantaneous reaction of the user 102 is content, the biological feedback detection model 28 determines that a normal user experience occurred in the application 22 based on determining that the user 102 is content. The biological feedback detection model 28 provides feedback 20 in response to determining that a normal user experience occurred in the application based on the analysis of the instantaneous reaction of the user 102.

The biological feedback detection model 28 is trained using the processed signals 18a, 18b to learn individual profiles of the user 102. For example, the processed signals 18a, 18b of the user 102 are correlated with the feedback 20 to learn personalized suggestions or improvements for the application 22 based on the user's 102 interactions with the application 22. The biological feedback detection model 28 may use the processed signals 18a, 18b in combination with the feedback 20 to learn a customized habit of the user 102 and provide a personalized user experience for the application 22 based on a current state of the user 102.

The feedback 20 provides the user experience classification 32 of the user's 102 interactions with the application 22. In some implementations, the feedback 20 indicates that an undesirable user experience occurred in the application 22. In some implementations, the feedback 20 indicates that a positive user experience occurred in the application 22. In some implementations, the feedback 20 indicates that a normal user experience occurred in the application 22. As such, the biological feedback detection model 28 receives time series of data of the different processed signals 18a, 18b as input and determines whether the application 22 provided a good functionality for the user interface or user experience of the application 22 or a bad functionality for the user interface or user experience of the application 22 based on the current state of the user 102. The feedback 20 provided by the biological feedback detection model 28 indicates the user's 102 instantaneous reaction (e.g., a positive reaction, a negative reaction, or an indifferent reaction) to the user interface or user experience of the application 22. As such, the feedback 20 captures the current state of the user 102 as the user 102 interacts with the user interface or the user experience of the application 22 based on an analysis of the biological feedback obtained from the user 102.

The feedback 20 is provided to a machine learning model 30 that provides the suggestions for the user interface or user experience of the application 22. The machine learning model 30 receives the feedback 20 as input and may provide revised suggestions or functionalities for the user interface or user experience as output for the application 22 based on the feedback 20. If the feedback 20 indicates that an undesirable user experience occurred in the application 22, the machine learning model 30 updates the suggestion or functionalities for the application 22 and provides a different or modified suggestion or functionality to the application 22. For example, the machine learning model 30 re-ranks the suggestions in response to the feedback 20 and provides different suggestions in response to the feedback 20. Another example includes the machine learning model 30 sending a suggestion to the application 22 to recall a sent e-mail message. Another example includes the machine learning model 30 sending a suggestion to the application 22 to undo a previous command, such as, deletion of text. In some implementations, the machine learning model 30 updates the recommendations provided to the application 22 in near real time in response to receiving the feedback 20. For example, the machine learning model 30 changes the recommendations or suggestions provided to the application 22 based on the feedback 20 indicating that the user 102 is having an unpleasant experience with the application 22. If the feedback 20 indicates a positive user experience or a normal user experience for the application 22, the machine learning model 30 may continue to provide the suggestions or functionalities to the application 22 without modification.

In some implementations, the machine learning model 30 is trained offline with a large set of data across multiple users. In some implementations, the machine learning model 30 is trained using the feedback 20. In some implementations, the machine learning model 30 is trained offline and online. In some implementations, the machine learning model 30 is a natural language processing mode (NPL). In some implementations, the machine learning model 30 operates on the device 104 of the user 102 (e.g., the device the user 102 is using to access the application 22). In some implementations, the machine learning model 30 is in a cloud server device remote from the device 104. In some implementations, the machine learning model 30 has components that are both local to the device 104 of the user 102 and remote from the device 104 of the user 102 (e.g., in a cloud server device).

As such, in near real time, the biological feedback detection model 28 determines a user experience classification 32 of the user's 102 interactions with the application 22 in response to the analysis of the processed signals 18a, 18b and provides the feedback 20 to the application 22 and/or the machine learning model 30 with the user experience classification 32.

In some implementations, the application 22 automatically corrects the user interface or user experience in response to the feedback 20 indicating an undesirable user experience of the application 22. If the feedback 20 indicates that an undesirable user experience occurred in the user experience or the user interface, the application 22 may automatically correct the user interface or the user experience or provide a different user interface or user experience to the user 102. For example, the application 22 changes a ranking of the autosuggestions provided to the user 102 in response to receiving the feedback 20 indicating that an undesirable user experience occurred in the application 22. Another example includes the application 22 recalling a sent e-mail message. Another example includes the application 22 undoing a previous command, such as, deletion of text.

By the application 22 automatically correcting the user interface or the user experience, the application 22 is more accessible to users with limited mobility or accessibility issues. The undesirable user experiences are automatically corrected by the application 22 in response to the feedback 20 without conventional user input. In addition, the user experience of the application 22 improves by automatically adapting or changing the user interface of the application 22 or the user experience of the application 22. As such, the biological feedback detection model 28 analyzes the processed signals 18a, 18b providing the biological feedback of the user 102 as the user 102 interacts with the application 22 and determines a user experience classification 32 of the application 22 in response to the analysis of the biological feedback of the user.

The actions (e.g., recalling an e-mail message, undoing a command, changing a user interface, etc.) the application 22 takes to correct or adapt the behavior of the application 22 in response to receiving the feedback 20 is provided to the machine learning model 30. The machine learning model 30 uses the user experience classification 32 and the actions taken by the application 22 to improve future recommendations or suggestions for the application 22 and reduce future errors in the recommendations or suggestions provided to the application 22.

In some implementations, the data storage 24 stores a user profile with the processed signals 18a, 18b and the user experience classification 32 of the application 22. The user profile is used to train the machine learning model 30 and/or the biological feedback detection model 28. In some implementations, the feedback 20 is provided to the data storage 24 to save with the user profile information. The machine learning model 30 may provide a customized user experience with the application 22 based on the user profile information learned from the biological feedback of the user 102 interacting with the application 22. Moreover, the user profile information in the data storage 24 may be used for providing targeted advertisements to the user 102 during use of the applications 22. In some implementations, the user 102 may opt out of having the user profile stored or user information is unavailable for the user 102. The machine learning model 30 may use offline training and/or online training of different base models to determine the user experience with the application 22. As such, the machine learning model 30 provides the user 102 with the user experience for the application 22 based on the training of the different base models.

In some implementations, one or more computing devices (e.g., servers and/or devices) are used to perform the processing of the environment 200. The one or more computing devices may include, but are not limited to, server devices, personal computers, a mobile device, such as, a mobile telephone, a smartphone, a PDA, a tablet, or a laptop, and/or a non-mobile device. The features and functionalities discussed herein in connection with the various systems may be implemented on one computing device or across multiple computing devices. For example, the application 22, the data storage 24, the signal processing component 26, the biological feedback detection model 28, and the machine learning model 30 are implemented wholly on the same computing device. Another example includes one or more subcomponents of the application 22, the data storage 24, the signal processing component 26, the biological feedback detection model 28, and the machine learning model 30 are implemented across multiple computing devices. Moreover, in some implementations, one or more subcomponent of the application 22, the data storage 24, the signal processing component 26, the biological feedback detection model 28, and the machine learning model 30 may be implemented are processed on different server devices of the same or different cloud computing networks.

In some implementations, each of the components of the environment 200 is in communication with each other using any suitable communication technologies. In addition, while the components of the environment 200 are shown to be separate, any of the components or subcomponents may be combined into fewer components, such as into a single component, or divided into more components as may serve a particular implementation. In some implementations, the components of the environment 200 include hardware, software, or both. For example, the components of the environment 200 may include one or more instructions stored on a computer-readable storage medium and executable by processors of one or more computing devices. When executed by the one or more processors, the computer-executable instructions of one or more computing devices can perform one or more methods described herein. In some implementations, the components of the environment 200 include hardware, such as a special purpose processing device to perform a certain function or group of functions. In some implementations, the components of the environment 200 include a combination of computer-executable instructions and hardware.

The environment 200 takes the user's 102 physiological and cognitive signals in an integrated as physiological sensing and provides personalized interactions with applications 22 based on the physiological sensing of the user 102. The environment 200 detects the user's 102 subtle reaction to behaviors of the application 22 from the biological feedback of the user 102 (e.g., the user's 102 physiological and cognitive signals) interacting with the application 22 and corrects unexpected behaviors of the application 22 or undesirable behaviors of the application 22 in response to detecting the user's 102 reaction. As such, the environment 200 improves the user's 102 experience with the application 22 based on the physiological sensing of the user 102 and allows personalized interactions with applications 22 by the user 102.

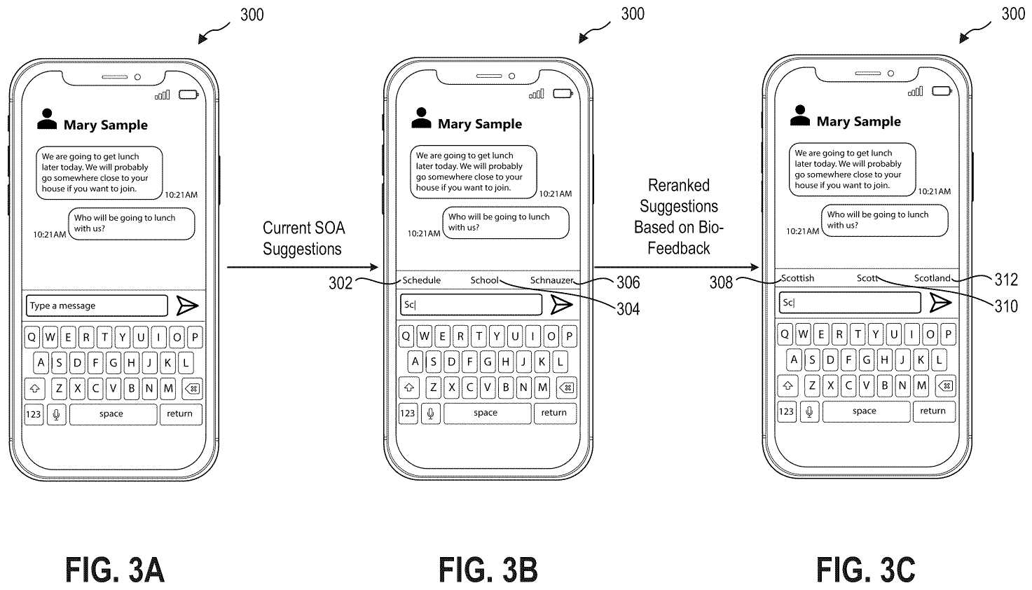

Referring now to FIG. 3A, illustrated is an example user interface 300 of an application 22 (FIG. 1) that the user 102 is using on a device 104 (FIG. 1) (e.g., a cellphone of the user 102). For example, the application 22 is a messaging application that the user 102 is using to message another individual about going to get lunch later today. The biological feedback of the user 102 is obtained as the user 102 is messaging about lunch using the application 22. For example, a camera on the device 104 obtains the facial expressions of the user 102 (e.g., the bodily signals 10 (FIG. 1)). In addition, a wearable EEG device (e.g., the neural device 106) obtains the neural signals 12 (FIG. 1) of the user 102. As such, a plurality of signals (e.g., the bodily signals 10 and the neural signals 12) are obtained in near real time as the user 102 is messaging about lunch using the application 22.

Referring now to FIG. 3B, the user interface 300 illustrates the suggestions (Schedule 302, School 304, and Schnauzer 306) provided by the application 22 for automatically replying to the question “Who will be going to lunch with us?” in response to the user 102 starting to type “Sc.” The biological feedback of the user is obtained (e.g., the bodily signals 10 using the camera and the neural signals 12 using the neural device 106) and the biological feedback detection model 28 (FIG. 2) receives the processed signals 18a, 18b based on the bodily signals 10 and the neural signals 12 obtained from the user 102. The biological feedback detection model 28 analyzes the processed signals 18a, 18b and determines that the user 102 is having an unexpected experience using the application 22. For example, the analysis of the neural signals 12 indicates surprise by the user 102. Another example includes the analysis of the facial expression of the user 102 indicates that the user 102 is puzzled. Another example includes the analysis of the heart rate variability (HRV) indicates that the user 102 is annoyed. The biological feedback detection model 28 determines that an undesirable user experience occurred with the suggestions provided by the application 22 and sends the feedback 20 (FIG. 2) to the application 22 with the user experience classification 32 of user's 102 interactions with the application 22 (e.g., an undesirable user experience occurred).

Referring now to FIG. 3C, the user interface 300 illustrates the revised suggestions (Scottish 308, Scott 310, Scotland 312) provided by the application 22 in response to receiving the feedback 20. The application 22 receives the feedback 20 indicating that an undesirable user experience occurred with the suggestions and automatically selects different suggestions in response to receiving the feedback 20. For example, the application 22 applies a different ranking to the suggestions based on the feedback 20.

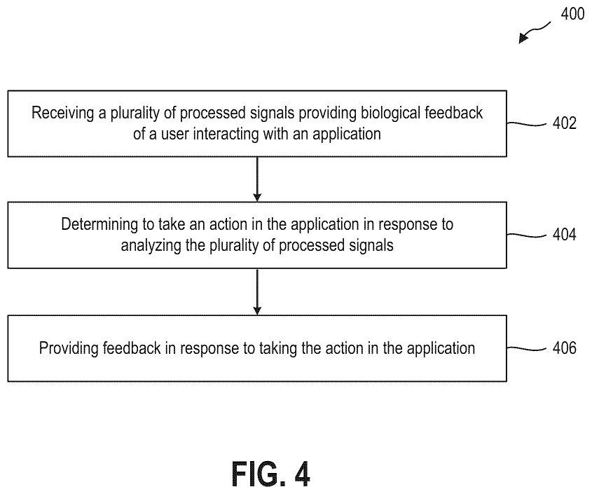

Referring now to FIG. 4, illustrated is an example method 400 for determining that an undesirable user experience occurred in an application. The actions of the method 400 are discussed below with reference to the architecture of FIGS. 1 and 2.

At 402, the method 400 includes receiving a plurality of processed signals providing biological feedback of a user interacting with an application. The biological feedback detection model 28 receives a plurality of processed signals 18a, 18b in near real time as the user 102 interacts with the application 22. The plurality of processed signals 18a, 18b provides the biological feedback of the user 102.

In some implementations, the plurality of processed signals 18a, 18b include the neural signals 12 of the user 102. In some implementations, the plurality of processed signals 18a, 18b include the bodily signals 10 of the user 102. In some implementations, the plurality of processed signals 18a, 18b include the physiological signals 14 of the user 102. In some implementations, the plurality of processed signals 18a, 18b include the neural signals 12 and the bodily signals 10 of the user 102. In some implementations, the plurality of processed signals 18a, 18b include the neural signals 12 and the physiological signals 14 of the user 102. In some implementations, the plurality of processed signals 18a, 18b include the bodily signals 10 and the physiological signals 14 of the user 102. In some implementations, the plurality of processed signals 18a, 18b include the neural signals 12, the bodily signals 10, and the physiological signals 14 of the user 102. As such, the plurality of processed signals 18a, 18b include any combination or sub-combination of the biological feedback obtained of the user 102 interacting with the application 22.

At 404, the method 400 includes determining to take an action in the application in response to analyzing the plurality of processed signals. The biological feedback detection model 28 determines that the user experience classification 32 of the user's 102 interaction with the application 22 is an undesirable user experience in response to analyzing the plurality of processed signals 18a, 18b. In some implementations, the plurality of processed signals 18a, 18b include features that indicate an instantaneous reaction of the user 102 with the application. The biological feedback detection model 28 identifies features in the plurality of processed signals 18a, 18b that indicate a current state of the user 102 and compares the current state of the user 102 to known states to determine an instantaneous reaction of the user 102 in response to encountering the undesirable user experience in the application 22. For example, the known states are frustration, surprise, agitation, anger, pleased, content, happy, or indifferent. The biological feedback detection model 28 uses the instantaneous reaction to identify that the undesirable user experience occurred in the user's 102 interactions with the application 22.

The application 22 receives the feedback 20 and determines that an action is necessary in response to determining that user experience classification 32 is an undesirable user experience. For example, the action includes denying auto-correct, changing lighting, recalling a sent e-mail message, correcting a command, moving scroll bar, selecting a button, or stopping a voice call. In some implementations, the application 22 receives the feedback 20 and determines that there is no valid or appropriate action for the application to take based on the feedback 20 indicating the undesirable user experience. The application 22 may provide additional feedback to the machine learning model 30 of the determination to take no action based on the feedback 20 indicating the undesirable user experience, which may enable future improvements of the application 22 by the machine learning model 30 modifying the suggestions or recommendations provided to the application 22.

At 406, the method 400 includes providing feedback in response to taking the action in the application. The biological feedback detection model 28 provides feedback 20 that the undesirable user experience occurred in the application 22. In some implementations, the undesirable user experience is an error in the user interface of the application 22. For example, a user interface error includes a scroll bar on the user interface not scrolling (e.g., the user tries to move the scroll bar on the user interface, and nothing happens in response to moving the scroll bar). Another example of a user interface error includes a button on the user interface not clicking (e.g., the user 102 selects the button and no action occurs in response to selecting the button). As such, a user interface error occurs in response to the application 22 making a functional error. In some implementations, the undesirable user experience is a user experience error of the application 22. The user experience error is an unexpected or bad interactions of the user 102 with the application 22. The user error experience error may occur even if the user interface is functional and performing correctly. For example, the application 22 provides an incorrect suggestion for a word to automatically fill into a message. Another example includes the user 102 accidentally sends a message.

In some implementations, the biological feedback detection model 28 provides the feedback 20 to the application 22. The application 22 provides a change in near real time to correct the user experience of the application 22 based on the feedback 20. In some implementations, the biological feedback detection model 28 provides the feedback 20 to a machine learning model 30 providing suggestions to the application 22. The machine learning model 30 provides a change in near real time to correct the undesirable user experience in the application 22 based on the feedback 20. For example, the machine learning model 30 provides different suggestions for the application 22 to provide to the user 102 based on the feedback 20. In some implementations, the machine learning model 30 provides a personalized experience of the application 22 to the user 102 based on the biological feedback of the user 102. For example, the machine learning model 30 provides personalized suggestions for the application 22 to the user 102 (e.g., different suggestions relative to other users of the application 22) based on the biological feedback of the user 102.

Additional feedback 20 is provided to the application 22 and the machine learning model 30 in response to taking the action in the application 22 to correct or adapt the behavior of the application 22 in response to the user experience classification 32 indicating that an undesirable user experience occurred in the application 22. The machine learning model 30 uses the additional feedback to improve system performance for future suggestions for the application 22.

As such, the method 400 is used to determine a user experience classification 32 of the user's 102 interactions with the application 22 based on the biological feedback of the user 102 as the user 102 interacts with the application 22. The method 400 is also used to adapt or change a behavior of the application 22 by taking different actions in the application 22 in response to determining that the user experience classification 32 is an undesirable user experience of the application 22. In addition, the method 400 is used to improve system performance by using the user experience classification 32 in the feedback 20 to improve the suggestions provided by the machine learning model 30 to the application 22.

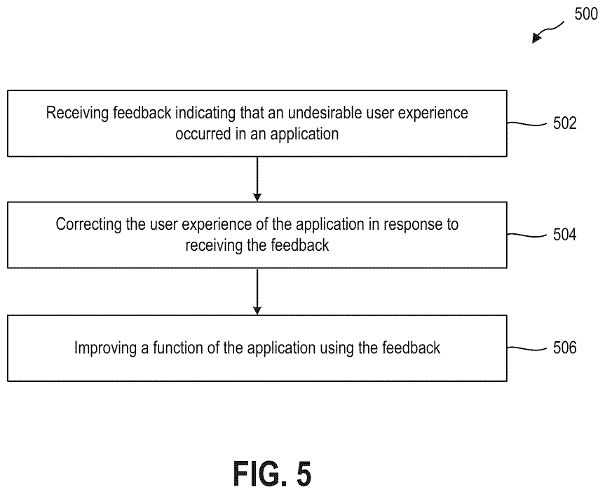

Referring now to FIG. 5, illustrated is an example method 500 for correcting user interface errors in an application. The actions of the method 500 are discussed below with reference to the architecture of FIGS. 1 and 2.

At 502, the method 500 includes receiving feedback indicating that an undesirable user experience occurred in an application, where the feedback is based on an analysis of biological feedback of the user interacting with the application. The application 22 receives the feedback 20 indicating that the user experience classification 32 of the application 22 is an undesirable user experience.

In some implementations, the biological feedback includes the neural signals 12 of the user 102 interacting with the application 22. In some implementations, the biological feedback includes the bodily signals 10 of the user 102 interacting with the application 22. In some implementations, the biological feedback includes the physiological signals 14 of the user 102 interacting with the application 22. In some implementations, the analysis of the biological feedback of the user 102 interacting with the application 22 indicates an instantaneous reaction of the user 102 in response to encountering the undesirable user experience in the application 22.

At 504, the method 500 includes correcting the user experience in response to receiving the feedback. In some implementations, the application 22 corrects the undesirable user experience in near real time in response to receiving the feedback 20. One example includes the application 22 automatically rejecting programming keywords, functions or variables, and thus, improving coding productivity of the user 102. Another example includes the application 22 rejecting erroneous auto fills in data entry programs improving the usability of the data entry programs. Another example includes the application 22 correcting a text completion or autocorrection in an e-mail to match a user's 102 expectation, and thus, improving a usability of the application 22 by the user 102. Another example includes the application 22 automatically recalling sent messages in response to the user 102 accidentally pushing the send button improving a user experience of using the application 22. Another example includes the application 22 reverting unwanted changes or deletion of text in a document application in response to a user mistakenly highlighting the wrong section of a piece of text or inserts content in an unexpected location improving the usability of the application 22.

At 506, the method 500 includes improving a function of the application using the feedback. The application 22 uses the feedback 20 to improve a function of the application 22 to reduce future errors in the user interface or the user experience of the application 22. In some implementations, the machine learning model 30 uses the feedback 20 to improve the suggestions or functionalities provided to the application 22 to future errors in the user experience of the application 22. As such, the application 22 uses the feedback 20 with the user experience classification 32 to reduce future errors and improve the performance of the application 22.

As such, the method 500 is used for correcting errors in the user interface of an application 22 based on the biological feedback of the user 102.

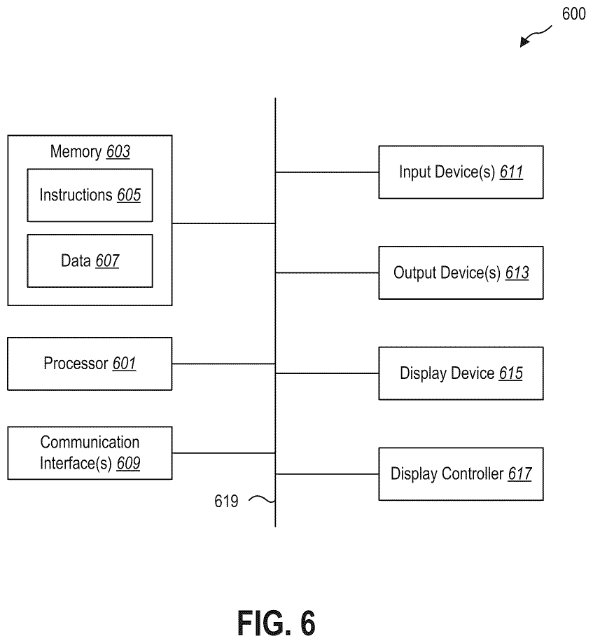

FIG. 6 illustrates components that may be included within a computer system 600. One or more computer systems 600 may be used to implement the various methods, devices, components, and/or systems described herein.

The computer system 600 includes a processor 601. The processor 601 may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 601 may be referred to as a central processing unit (CPU). Although just a single processor 601 is shown in the computer system 600 of FIG. 6, in an alternative configuration, a combination of processors (e.g., an ARM and DSP) could be used.

The computer system 600 also includes memory 603 in electronic communication with the processor 601. The memory 603 may be any electronic component capable of storing electronic information. For example, the memory 603 may be embodied as random access memory (RAM), read-only memory (ROM), magnetic disk storage mediums, optical storage mediums, flash memory devices in RAM, on-board memory included with the processor, erasable programmable read-only memory (EPROM), electrically erasable programmable read-only memory (EEPROM) memory, registers, and so forth, including combinations thereof.

Instructions 605 and data 607 may be stored in the memory 603. The instructions 605 may be executable by the processor 601 to implement some or all of the functionality disclosed herein. Executing the instructions 605 may involve the use of the data 607 that is stored in the memory 603. Any of the various examples of modules and components described herein may be implemented, partially or wholly, as instructions 605 stored in memory 603 and executed by the processor 601. Any of the various examples of data described herein may be among the data 607 that is stored in memory 603 and used during execution of the instructions 605 by the processor 601.

A computer system 600 may also include one or more communication interfaces 609 for communicating with other electronic devices. The communication interface(s) 609 may be based on wired communication technology, wireless communication technology, or both. Some examples of communication interfaces 609 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system 600 may also include one or more input devices 611 and one or more output devices 613. Some examples of input devices 611 include a keyboard, mouse, microphone, remote control device, button, joystick, trackball, touchpad, and lightpen. Some examples of output devices 613 include a speaker and a printer. One specific type of output device that is typically included in a computer system 600 is a display device 615. Display devices 615 used with embodiments disclosed herein may utilize any suitable image projection technology, such as liquid crystal display (LCD), light-emitting diode (LED), gas plasma, electroluminescence, or the like. A display controller 617 may also be provided, for converting data 607 stored in the memory 603 into text, graphics, and/or moving images (as appropriate) shown on the display device 615.

The various components of the computer system 600 may be coupled together by one or more buses, which may include a power bus, a control signal bus, a status signal bus, a data bus, etc. For the sake of clarity, the various buses are illustrated in FIG. 6 as a bus system 619.

In some implementations, the various components of the computer system 600 are implemented as one device. For example, the various components of the computer system 600 are implemented in a mobile phone or tablet. Another example includes the various components of the computer system 600 implemented in a personal computer.

As illustrated in the foregoing discussion, the present disclosure utilizes a variety of terms to describe features and advantages of the model evaluation system. Additional detail is now provided regarding the meaning of such terms. For example, as used herein, a “machine learning model” refers to a computer algorithm or model (e.g., a classification model, a clustering model, a regression model, a language model, an object detection model) that can be tuned (e.g., trained) based on training input to approximate unknown functions. For example, a machine learning model may refer to a neural network (e.g., a convolutional neural network (CNN), deep neural network (DNN), recurrent neural network (RNN)), or other machine learning algorithm or architecture that learns and approximates complex functions and generates outputs based on a plurality of inputs provided to the machine learning model. As used herein, a “machine learning system” may refer to one or multiple machine learning models that cooperatively generate one or more outputs based on corresponding inputs. For example, a machine learning system may refer to any system architecture having multiple discrete machine learning components that consider different kinds of information or inputs.

The techniques described herein may be implemented in hardware, software, firmware, or any combination thereof, unless specifically described as being implemented in a specific manner. Any features described as modules, components, or the like may also be implemented together in an integrated logic device or separately as discrete but interoperable logic devices. If implemented in software, the techniques may be realized at least in part by a non-transitory processor-readable storage medium comprising instructions that, when executed by at least one processor, perform one or more of the methods described herein. The instructions may be organized into routines, programs, objects, components, data structures, etc., which may perform particular tasks and/or implement particular data types, and which may be combined or distributed as desired in various implementations.

Computer-readable mediums may be any available media that can be accessed by a general purpose or special purpose computer system. Computer-readable mediums that store computer-executable instructions are non-transitory computer-readable storage media (devices). Computer-readable mediums that carry computer-executable instructions are transmission media. Thus, by way of example, and not limitation, implementations of the disclosure can comprise at least two distinctly different kinds of computer-readable mediums: non-transitory computer-readable storage media (devices) and transmission media.

As used herein, non-transitory computer-readable storage mediums (devices) may include RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

The steps and/or actions of the methods described herein may be interchanged with one another without departing from the scope of the claims. In other words, unless a specific order of steps or actions is required for proper operation of the method that is being described, the order and/or use of specific steps and/or actions may be modified without departing from the scope of the claims.

The term “determining” encompasses a wide variety of actions and, therefore, “determining” can include calculating, computing, processing, deriving, investigating, looking up (e.g., looking up in a table, a database, a datastore, or another data structure), ascertaining and the like. Also, “determining” can include receiving (e.g., receiving information), accessing (e.g., accessing data in a memory) and the like. Also, “determining” can include resolving, selecting, choosing, establishing, predicting, inferring, and the like.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one implementation” or “an implementation” of the present disclosure are not intended to be interpreted as excluding the existence of additional implementations that also incorporate the recited features. For example, any element described in relation to an implementation herein may be combinable with any element of any other implementation described herein. Numbers, percentages, ratios, or other values stated herein are intended to include that value, and also other values that are “about” or “approximately” the stated value, as would be appreciated by one of ordinary skill in the art encompassed by implementations of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to implementations disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the implementations that falls within the meaning and scope of the claims is to be embraced by the claims.

The present disclosure may be embodied in other specific forms without departing from its spirit or characteristics. The described implementations are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method, comprising:
receiving a plurality of processed signals providing biological feedback of a user interacting with an application;
determining to take an action in the application in response to analyzing the plurality of processed signals; and
providing feedback in response to taking the action in the application.

2. The method of claim 1, wherein the plurality of processed signals include neural signals of the user.

3. The method of claim 1, wherein the plurality of processed signals include bodily signals of the user.

4. The method of claim 1, wherein the plurality of processed signals include physiological signals of the user.

5. The method of claim 1, wherein the plurality of processed signals include neural signals of the user and bodily signals of the user.

6. The method of claim 1, wherein the plurality of processed signals include features that indicate an instantaneous reaction of the user with the application.

7. The method of claim 1, wherein determining to take an action in the application in response to analyzing the plurality of processed signals further comprises:
identifying features in the plurality of processed signals that indicate a current state of the user;
comparing the current state of the user to known states to determine an instantaneous reaction of the user interacting with the application; and
using the instantaneous reaction to identify that an undesirable user experience occurred in the application.

8. The method of claim 7, wherein the known states are one or more of frustration, surprise, agitation, anger, pleased, content, happy, or indifferent.

9. The method of claim 7, wherein the undesirable user experience is a sub-optimal state of a user interface of the application.

10. The method of claim 7, wherein the undesirable user experience is a sub-optimal state in a user experience of the application.

11. The method of claim 1, wherein the feedback is provided to the application in near real time with positive feedback or negative feedback.

12. The method of claim 1, wherein the feedback is provided to a machine learning model providing functionality to the application.

13. The method of claim 1, further comprising:
providing a change in near real time to correct an undesirable user experience in the application based on the feedback.

14. The method of claim 1, further comprising:
providing a personalized experience of the application to the user based on the biological feedback of the user.

15. The method of claim 1, further comprising:
providing additional feedback to a machine learning model based on the biological feedback of the user to improve future performance of the machine learning model.

16. A system, comprising:
a processor;
memory in electronic communication with the processor; and
instructions stored in the memory, the instructions being executable by the processor to:
receive feedback indicating that an undesirable user experience occurred in an application, wherein the feedback is based on an analysis of biological feedback of a user interacting with the application; and
correct the user experience of the application in response to receiving the feedback.

17. The system of claim 16, wherein the analysis of biological feedback of the user interacting with the application indicates an instantaneous reaction of the user in response to encountering the undesirable user experience in the application; and
the instructions are further executable by the processor to correct the undesirable user experience in near real time in response to receiving the feedback.

18. The system of claim 16, wherein the biological feedback includes neurological signals of the user interacting with the application.

19. The system of claim 16, wherein the biological feedback includes bodily signals of the user interacting with the application.

20. The system of claim 16, wherein the biological feedback includes physiological signals of the user interacting with the application.
